(19) United States
(12) Patent Application Publication
(10) Pub. No.: US 2007/0267024 A1
(43) Pub. Date: Nov. 22, 2007

(54) Kremer's Oral Airway

(75) Inventors: Anatoly Kremer, Huntingdon Valley, PA (US); Boris Primak, Brooklyn, NY (US); Alexander Fridman, Yardley, PA (US)

Correspondence Address: DAVID H. SIEGEL, 315 E 72ND ST. 34E, NEW YORK, NY, 10021, US

(73) Assignees: Mr. Anatoly Kremer, Huntingdon Valley, PA (US); Mr. Boris Primak, Brooklyn, NY (US); Mr. Alexander Fridman, Yardley, PA (US)

(21) Appl. No.: 10/905,731

(22) Filed: Jan. 19, 2005

(51) Int. Cl. A61M 16/00; A62B 9/06

(52) U.S. Cl. 128/207.14; 128/200.26

(57) ABSTRACT

An oral airway device comprising an outer channel housing an oxygen delivery tube and a carbon dioxide exhalation tube connected to the interior of the channel. Channel is shaped for insertion via mouth and to its internal end adjacent to the oropharynx when inserted. A bite block is attached to the channel and positioned between the internal and external ends of the channel for proper positioning of the channel when inserted. External end of channel includes oxygen delivery adaptor on the end of the oxygen delivery tube for connection to external oxygen source, and carbon dioxide monitoring adaptor on the end of the carbon dioxide exhalation tube for connection to carbon dioxide monitoring device.

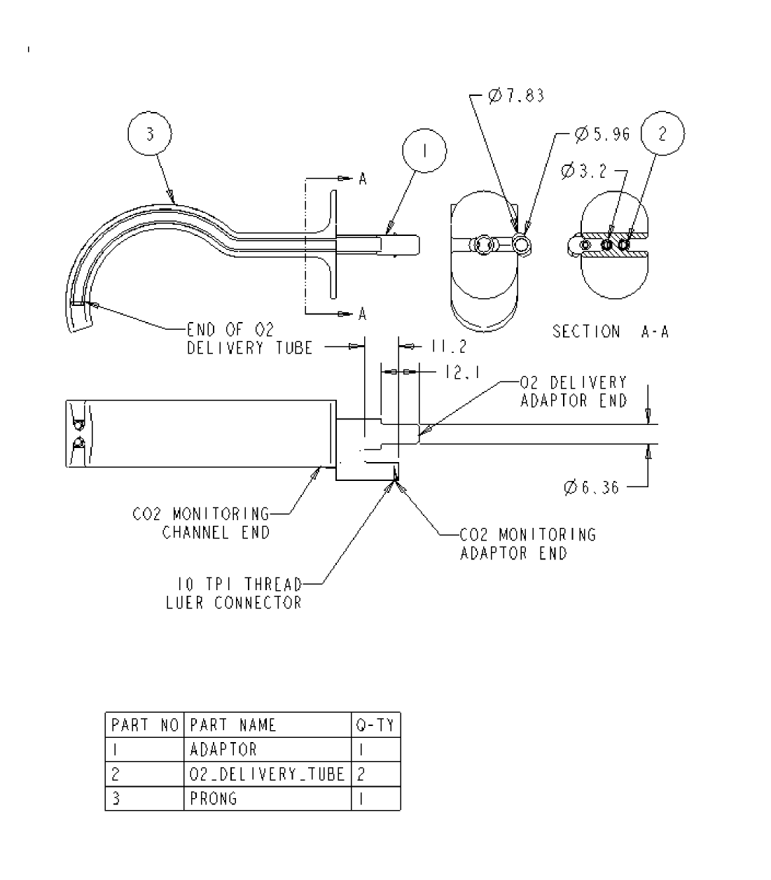

Original airway with capability to delivering of O₂ from outer source to oropharynx and simultaneous monitoring of exhaled CO₂ without risk of injury of delicate tissues as it has place in case of using of conventional airways.

The aforementioned Kremer's Oral Airway has several additional features that distinguish it from the airway types [Guedel or Berman] that are currently available on the market. Those features are.

What is claimed is:

1. Channel(s) for O₂ supply from outer source to oropharynx. These channel(s) is/are located parallel to axis of airway and is constructively connected with the airway's body. The entrance of the channel(s) is located in the plane of airway flanges and the open exit of this channel(s) is located just before the end of pharyngeal curve of airway.

2. The entrance of the channel(s) for the O₂ supply has entry connectors for the connection to the O₂ supplying tubes. The outer end of the connectors does not protrude beyond the plane of airway flanges.

3. A separate CO₂ tube with connector for connection with sampling tube-for constant monitoring of end-tidal CO₂. The open end of the tube is directed towards the oropharynx and the tube is built into the airway body and positioned in such a way as not to interfere with the other functions of the airway and at the same time provides uninterrupted sampling of exhaled air. The open end of the CO₂ tube is located just before the end of the pharyngeal curve. The connector of the CO₂ is located such as not to interfere with the functions of the airway and insure unobstructed access to connect the CO₂ sampling tube from the CO₂ monitor;
Existing oral airway are used to alleviate obstruction of upper airway [partial or complete] in the unconscious patient, or as a bite block, and also may be used as an adjunct for oral pharyngeal suctioning. There are several types of oral airways. The most often used are two types—Guedel and Berman. All of them have a flange to prevent swallowing and over insertion, a straight bite block portion, and a distal semicircular section to follow the curvature of the mouth, tongue, and posterior pharynx so that the tongue is displaced anteriorly. All type of oral airways has channel(s) connecting oropharynx with the atmosphere. The patient can breathe spontaneously with room air, or supplemental O₂ can be delivered by nasal canula [either simple or having additional channel for CO₂ monitoring]. Nasal and oral tissues are delicate and are subject to ease injury by conventional airways which potentially can damage structures. When using the current devices [conventional oral airways and nasal canula] there are several shortcomings:
a) Nasal canula is not effective in delivering O₂ to the patient because it requires open nasal passages and cannot achieve FIO₂ above 0.44;
b) O₂ flow can cause mucosal drying which may result in mucosal irritation, nosebleeds, earache, bronchospasm, etc;
c) Mechanical pressure from nasal prongs of canula may cause tissue damage;
d) If the nasal canula has CO₂ monitoring channel results may be inaccurate if the patient does not breathe through the nose or nasal passages are obstructed.
